(19) United States
(12) Patent Application Publication
(10) Pub. No.: US 2025/0242347 A1
(43) Pub. Date: Jul. 31, 2025

(54) MICROPARTICLE SORTING DEVICE AND MICROPARTICLE SORTING KIT

(71) Applicant: SONY GROUP CORPORATION, TOKYO (JP)

(72) Inventor: SOH NISHIMURA, TOKYO (JP)

(21) Appl. No.: 18/702,887

(22) PCT Filed: Oct. 21, 2022
(86) PCT No.: PCT/JP2022/039237

§ 371 Date: Apr. 19, 2024

(30) Foreign Application Priority Data
Oct. 29, 2021 (JP) 2021-177145

(51) Int. Cl. B01L 3/00

(52) U.S. Cl. CPC B01L 3/502761; B01L 2200/0652; B01L 2300/0654

(57) ABSTRACT

Provided are a microparticle sorting device and a microparticle sorting kit capable of simplifying work related to changing a sample liquid. The present technology provides a microparticle sorting device including: a kit attachment surface to which a microparticle sorting kit is detachably attached; a storage portion including a top plate portion having a top plate opening and a lid plate portion having a first surface and capable of opening and closing the top plate opening, the storage portion having a space therein; a first holding portion capable of holding a sample liquid accommodation container configured to accommodate a sample liquid containing microparticles; and a second holding portion capable of holding a container, in which the first holding portion is provided in the space of the storage portion, the second holding portion is provided on the first surface of the lid plate portion, and the lid plate portion is capable of closing the top plate opening with the first surface and the second holding portion facing the space of the storage portion. The present technology further provides the microparticle sorting kit.

### TECHNICAL FIELD

The present technology relates to a microparticle sorting device and a microparticle sorting kit.

### BACKGROUND ART

As a method for sorting a target biological sample from a specific biological sample, a membrane separation method, a centrifugal separation method, an electrical separation method, a method for killing biological samples other than the target biological sample, a magnetic bead method for separating the target biological sample by labeling the target biological sample with magnetic beads, flow cytometry, and the like are known. Examples of a technology related to the flow cytometry include a microparticle sorting device using a microchip disclosed in the following Patent Document 1.

A so-called flow cytometer as disclosed in the following Patent Document 1 may cause droplets to fly in a space, so that there has been a possibility that the flow cytometer and its surrounding environment are contaminated by a mist containing a biological sample being sorted. Furthermore, a sorting mechanism is exposed to an external atmosphere, so that there has also been a possibility that another substance in the external atmosphere is mixed into the biological sample that has been sorted. It is therefore difficult to use the flow cytometer for immune cell therapy and the like.

Therefore, proposed is a sample sorting kit and a sample sorting device capable of sorting and collecting a target biological sample in an enclosed space. For example, the following Patent Document 2 discloses a sample sorting kit including an accommodation portion that accommodates a sample being sorted, a sorting portion that sorts a target biological sample from the sample being sorted, and a collection portion that accommodates the target biological sample, and the accommodation portion, the sorting portion, and the collection portion are airtightly connected, and a sample sorting device including the sample sorting kit.

### CITATION LIST

### Patent Document

Patent Document 1: Japanese Patent Application Laid-Open No. 2011-237201

Patent Document 2: Japanese Patent Application Laid-Open No. 2017-181278

### SUMMARY OF THE INVENTION

### Problems to be Solved by the Invention

The sorting device capable of performing the sorting process in the enclosed space as disclosed in Patent Document 2 is suitable for manufacture of cell therapy drugs, for example, and can be used by an operator who manufactures cell therapy drugs in a pharmaceutical factory. Setting conditions of the sorting device when the operator manufactures the cell therapy drugs are predetermined by a researcher who is responsible for process development. In order to determine the setting conditions, the researcher needs to operate the sorting device using a plurality of sample liquids and verify the setting conditions. In a case where the researcher performs the verification using a plurality of sample liquids with a kit and a device that are capable of performing sorting in an enclosed space, after performing the verification using one sample liquid, the researcher changes the sample liquid to another sample liquid and then further performs the verification using the sample liquid. There is a possibility that the work to change a sample liquid is repeated many times, so that it is desirable to take as little time and effort as possible.

It is therefore a main object of the present technology to provide a microparticle sorting device and a microparticle sorting kit capable of simplifying the work related to changing a sample liquid.

### Solutions to Problems

That is, the present technology provides
a microparticle sorting device including:
a kit attachment surface to which a microparticle sorting kit is detachably attached;
a storage portion including a top plate portion having a top plate opening and a lid plate portion having a first surface and capable of opening and closing the top plate opening, the storage portion having a space therein;
a first holding portion capable of holding a sample liquid accommodation container configured to accommodate a sample liquid containing microparticles; and
a second holding portion capable of holding a container, in which
the first holding portion is provided in the space of the storage portion,
the second holding portion is provided on the first surface of the lid plate portion, and the lid plate portion is capable of closing the top plate opening with the first surface and the second holding portion facing the space of the storage portion.

In the microparticle sorting device, the lid plate portion may be separable from the top plate opening.

In the microparticle sorting device, the first holding portion may be detachably provided in the space of the storage portion.

The microparticle sorting device may further include a sample liquid stirring device located in the space of the storage portion, in which the first holding portion may be provided in the sample liquid stirring device, and the sample liquid stirring device may include a rotation portion configured to cause the first holding portion to perform a horizontal circular motion.

In the microparticle sorting device, the first holding portion may be detachably provided in the sample liquid stirring device.

In the microparticle sorting device, the second holding portion may be capable of holding a plurality of the containers.

In the microparticle sorting device, the second holding portion may be capable of holding a plurality of the containers having different capacities.

In the microparticle sorting device, the microparticle sorting kit may include a microchip, the microchip including: a sample liquid inlet into which the sample liquid containing the microparticles accommodated in the sample liquid accommodation container is introduced; a main channel through which the sample liquid that has been introduced flows; and a sorting channel where target microparticles are sorted from the sample liquid flowing through the main channel.

In the microparticle sorting device, the microparticle sorting kit may be configured to allow the sample liquid accommodation container to continuously connect to the microchip in a detachable manner.

In the microparticle sorting device, the microparticle sorting kit may be configured to allow a target sample collection portion to continuously connect to the microchip in a detachable manner, the target sample collection portion being configured to collect the target microparticles that have been sorted.

In the microparticle sorting device, the microparticle sorting kit may further include a pre-sample accommodation portion configured to accommodate the sample liquid and located upstream of the sample liquid accommodation container, and have no filter portion between the pre-sample accommodation portion and the sample liquid accommodation container.

In the microparticle sorting device, the microparticle sorting kit may have no filter portion between the sample liquid accommodation container and the microchip.

The microparticle sorting device may further include: a chip insertion unit into which the microchip is inserted; a light irradiation unit configured to irradiate the microparticles flowing through the main channel with light; a light detection unit configured to detect scattered light and/or fluorescence emitted from the microparticles; and a control unit configured to control a traveling direction of the microparticles flowing through the main channel on the basis of data detected by the light detection unit.

The microparticles may include bioparticles.

The bioparticles may include cells.

The present technology further provides
a microparticle sorting kit including a microchip, the microchip including: a sample liquid inlet into which a sample liquid containing microparticles accommodated in a sample liquid accommodation container is introduced; a main channel through which the sample liquid that has been introduced flows; and a sorting channel where target microparticles are sorted from the sample liquid flowing through the main channel,
the microparticle sorting kit being configured to allow the sample liquid accommodation container to continuously connect to the microchip in a detachable manner.

The microparticle sorting kit may be configured to allow a target sample collection portion to continuously connect to the microchip in a detachable manner, the target sample collection portion being configured to collect the target microparticles that have been sorted.

The microparticle sorting kit may further include a pre-sample accommodation portion configured to accommodate the sample liquid and located upstream of the sample liquid accommodation container, and have no filter portion between the pre-sample accommodation portion and the sample liquid accommodation container.

The microparticle sorting kit may have no filter portion between the sample liquid accommodation container and the microchip.

### BRIEF DESCRIPTION OF DRAWINGS

FIG. 1 is a schematic diagram illustrating an example of a microparticle sorting kit according to a first embodiment.

FIG. 2 is a schematic diagram illustrating an example of a sample liquid accommodation container.

FIG. 3 is a cross-sectional view illustrating a lid body and the periphery thereof in a state where the sample liquid accommodation container is turned upside down.

FIG. 4 is a schematic diagram illustrating an example of a microchip.

FIG. 5 is a schematic diagram illustrating an example of a microparticle sorting kit according to a modification of the first embodiment.

FIG. 6 is a schematic diagram illustrating an example of a microparticle sorting device according to a second embodiment.

FIG. 7 is a schematic diagram illustrating a part of a storage portion with a top plate opening in an open state.

FIG. 8 is a schematic diagram illustrating a part of the storage portion with the top plate opening in a closed state.

FIG. 9 is a schematic diagram illustrating a lid plate portion, a first holding portion, and a second holding portion.

FIG. 10 is a schematic diagram illustrating an example of a sample liquid stirring device.

FIG. 11 is a schematic diagram illustrating some of the components included in the microparticle sorting device.

### MODE FOR CARRYING OUT THE INVENTION

Hereinafter, preferred modes for carrying out the present technology will be described with reference to the drawings. The embodiments described below illustrate representative embodiments of the present technology, and the scope of the present technology is not limited only to these embodiments. The present technology will be described in the following order.

1. First embodiment (microparticle sorting kit)
(1) Overall configuration
(2) Configuration of sample liquid accommodation container
(3) Configuration of microchip
(4) Other configurations
(5) Modifications

2. Second embodiment (microparticle sorting device)
(1) Overall configuration
(2) Configuration of storage portion
(3) Other configurations

### 1. FIRST EMBODIMENT (MICROPARTICLE SORTING KIT)

### (1) Overall Configuration

A microparticle sorting kit according to a first embodiment of the present technology will be described. The microparticle sorting kit according to the first embodiment may be used with the microparticle sorting kit attached to a microparticle sorting device according to a second embodiment of the present technology described later, for example.

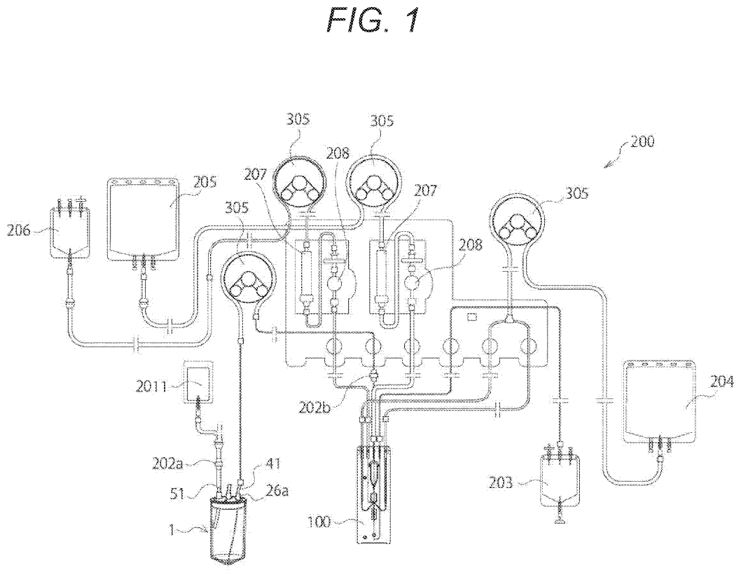

An overall configuration of a microparticle sorting kit 200 according to the first embodiment will be described with reference to FIG. 1. FIG. 1 is a schematic diagram illustrating an example of the microparticle sorting kit 200 according to the first embodiment.

The microparticle sorting kit 200 includes a microchip 100, the microchip 100 including a sample liquid inlet into which a sample liquid containing microparticles accommodated in a sample liquid accommodation container 1 is introduced, a main channel through which the sample liquid thus introduced flows, and a sorting channel where target microparticles are sorted from the sample liquid that has flowed through the main channel.

The microparticle sorting kit 200 is configured to allow the sample liquid accommodation container 1 to continuously connect to the microchip 100 in a detachable manner. In the present specification, the phrase “continuously connect” means that connection is established to allow the sample liquid to flow through. The microparticle sorting kit 200 may include one or a plurality of communication members for establishing continuous connection, that is, for allowing the sample liquid to flow through. Examples of the communication member include a channel (for example, a tube) through which the sample liquid flows, a coupling member that couples channels, and the like. Furthermore, in the present specification, the phrase “continuously connect in a detachable manner” means that connection is established to allow the sample liquid to flow through, and the connected portion can be separated.

For example, the microparticle sorting kit 200 may be configured to allow the sample liquid accommodation container 1 to continuously connect, in a detachable manner, to the microchip 100 (particularly, the sample liquid inlet of the microchip 100) via one or a plurality of communication members. In a state where the sample liquid accommodation container 1 is continuously connected to the microchip 100 via the one or plurality of communication members, the sample liquid can be supplied from the sample liquid accommodation container 1 to the microchip 100. For example, when the one or plurality of communication members is separated from the sample liquid accommodation container 1, the sample liquid accommodation container 1 is removed from the microparticle sorting kit 200.

For example, the sample liquid accommodation container 1 may be detachable between a lid body (specifically, an outer cylindrical portion 26a provided on the lid body) of the sample liquid accommodation container 1 and an outflow channel (specifically, a first outflow channel 41). That is, the sample liquid accommodation container 1 can be attached, for example, by connecting the outer cylindrical portion 26a to the first outflow channel 41, or can be removed by separating the outer cylindrical portion 26a from the first outflow channel 41.

The microparticle sorting kit 200 may or need not include the sample liquid accommodation container 1. That is, the sample liquid accommodation container 1 may or need not belong to a set of components constituting the microparticle sorting kit 200. In a case where the microparticle sorting kit 200 does not include the sample liquid accommodation container 1, for example, the user acquires, as necessary, the sample liquid accommodation container 1 to be continuously connected to the microparticle sorting kit 200. That is, the sample liquid accommodation container 1 may be prepared separately from the microparticle sorting kit 200 by the user.

In the microparticle sorting kit 200, the sample liquid accommodation container 1 is detachable. In a case where the user desires to change the sample liquid, the user only needs to replace the sample liquid accommodation container 1 accommodating the sample liquid, and need not replace the microparticle sorting kit 200. The use of the microparticle sorting kit 200 according to the present embodiment allows simplification of work related to replacement of the sample liquid and cost reduction as compared with a case where it is necessary to replace the kit when changing the sample liquid.

The microparticle sorting kit 200 is useful, for example, in a case where speedup and cost reduction of verification work are required rather than foreign matter contamination prevention when performing work to sort microparticles. For example, in a pharmaceutical factory that manufactures cell therapy drugs, when a researcher who is responsible for process development verifies setting conditions of the sorting device, there is a tendency that speedup and cost reduction of the verification work are required rather than foreign matter contamination prevention. Therefore, the microparticle sorting kit 200 is useful, for example, in a scene where the setting conditions of the sorting device are verified.

### (2) Configuration of Sample Liquid Accommodation Container

### (2-1) Overall Configuration

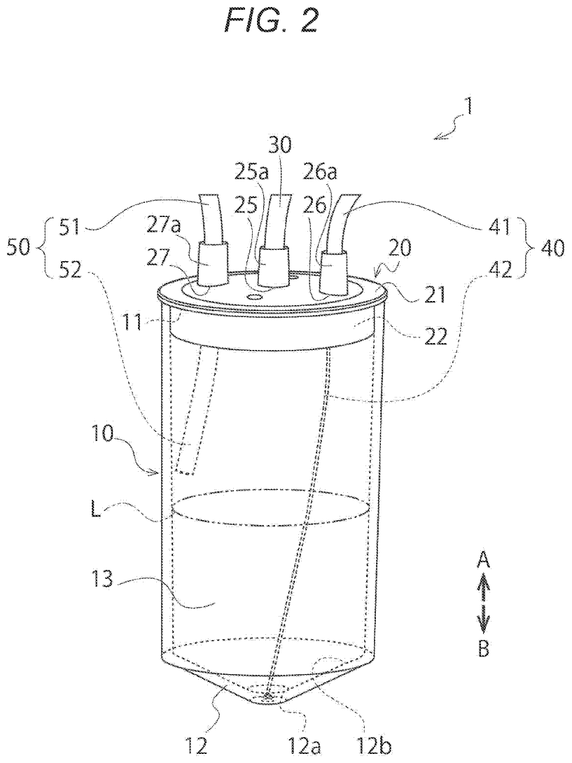

An overall configuration of the sample liquid accommodation container 1 will be described with reference to FIG. 2. FIG. 2 is a schematic diagram illustrating an example of the sample liquid accommodation container 1. The sample liquid accommodation container 1 includes a container main body 10 and a lid body 20. The container main body 10 has a bottomed cylindrical shape with an opening 11 at one end and a bottom surface portion 12 at the other end, and accommodates a sample liquid containing microparticles. The lid body 20 has a top surface portion 21, and seals the opening 11 of the container main body 10. In FIG. 2, an arrow A indicates a top surface direction of the sample liquid accommodation container 1, and an arrow B indicates a bottom surface direction of the sample liquid accommodation container 1.

The top surface portion 21 of the lid portion 20 may have a first through hole 25, a second through hole 26, and a third through hole 27. The sample liquid accommodation container 1 may include a vent pipe 30 connected to the first through hole 25. The sample liquid accommodation container 1 may include an outflow channel 40 that is connected to the second through hole 26 to allow the sample liquid to flow out from the container main body 10. The sample liquid accommodation container 1 may include an inflow channel 50 that is connected to the third through hole 27 to allow the sample liquid to flow into the container main body 10.

An example of a flow of the sample liquid in the microparticle sorting kit according to the present embodiment will be described below.

The sample liquid before being accommodated in the sample liquid accommodation container 1 may be accommodated in, for example, a pre-sample accommodation portion (for example, a pre-sample accommodation portion 2011 illustrated in FIG. 1) provided in the microparticle sorting kit. The sample liquid accommodated in the pre-sample accommodation portion flows into the container main body 10 through the inflow channel 50. The sample liquid accommodation container 1 is shaken, and thus the sample liquid in the container main body 10 is stirred. With this arrangement, the microparticles in the sample liquid are dispersed. The sample liquid flows out from the container main body 10 through the outflow channel 40. The sample liquid is caused to flow into and out from the container main body 10 by, for example, driving of a pump included in the microparticle sorting kit.

### (2-2) Configuration of Container Main Body

Next, the configuration of the container main body 10 will be described with reference to FIG. 2. As described above, the container main body 10 has a bottomed cylindrical shape with the opening 11 at one end and the bottom surface portion 12 at the other end. The portion between the opening 11 and the bottom surface portion 12 is a body portion 13, and the body portion 13 has a cylindrical shape.

The cylindrical shape of the container main body 10 allows, in a case where the container main body 10 is rotationally shaken, vibration generated by the rotational shaking to be transmitted better to the sample liquid. When the vibration is transmitted to the sample liquid, the sample liquid rotates. When the sample liquid rotates, the liquid level of the sample liquid becomes lower at the center and higher on the outside in the container main body 10 (that is, there is a difference in water level between the center and the outside), and this causes a difference in pressure (difference in weight) in the sample liquid. With this arrangement, a force due to a pressure gradient (that is, a centripetal force) is generated toward the center of the container main body 10. In the vicinity of the bottom surface portion 12 of the container main body 10, due to friction between the bottom surface portion 12 and the sample liquid, the centripetal force directed toward the center becomes larger than a centrifugal force directed outward. Thus, due to the centripetal force, the sample liquid moves toward the center in the vicinity of the bottom surface portion 12, and then is blown up. Thereafter, the sample liquid moves toward the outside by the centrifugal force, and flows down again toward the bottom surface portion 12. A secondary flow (flow in the longitudinal direction of the container main body 10) due to such centrifugal force and centripetal force causes the sample liquid to be effectively stirred and the microparticles to swirl up, and, as a result, the microparticles are favorably dispersed. That is, the cylindrical shape of the container main body 10 allows for favorable dispersibility of the microparticles in the sample liquid when the sample liquid is stirred.

When the microparticles in the sample liquid is excellent in dispersibility, it is possible to make the concentration of the sample liquid in the sample liquid accommodation container 1 uniform to make the concentration of the sample liquid fed from the sample liquid accommodation container 1 almost constant. With this arrangement, highly concentrated sample liquid can be prevented from flowing in the microparticle sorting kit. As a result, a problem that the sorting process is not in time and the sample liquid is wasted and a problem that clogging occurs in a liquid feeding tube and the sample liquid cannot be stably fed are less likely to occur.

Here, types of the microparticle sorting device include a closed-type device that sorts microparticles in a closed space and an open-type device that sorts microparticles in an open space. This “closed” means that fluid communication with an external environment is not performed. Since the sample liquid accommodation container 1 is detachable, the microparticle sorting kit according to the present embodiment can be used for an open-type microparticle sorting device. The configuration of the sample liquid accommodation container 1 described above, however, is also suitable for a closed-type microparticle device for the following reasons.

In a closed-type microparticle sorting device, it is necessary to stir a sample liquid in a sample liquid accommodation container without using stirring means (e.g., a stirring rod) brought from outside. Thus, it is desirable that the sample liquid used in the closed-type microparticle sorting device is stirred by rotational shaking type stirring that does not require external stirring means. That is, the sample liquid accommodation container used in the closed-type microparticle sorting device is desirably rotationally shaken.

As described above, in the sample liquid accommodation container 1, the microparticles in the sample liquid are favorably dispersed by rotational shaking. The sample liquid accommodation container 1 is therefore suitable for rotational shaking type stirring. That is, the sample liquid accommodation container 1 is also suitable for a closed-type microparticle sorting device.

The container main body 10 of the sample liquid accommodation container 1 will be continuously described with reference to FIG. 2. The inner surface of the bottom surface portion 12 of the container main body 10 preferably has a deepest portion 12a where the depth of the container main body 10 is greatest, and an inclined surface 12b inclined downward toward the deepest portion 12a. In the present specification, the “inner surface of the bottom surface portion” is a surface located inside the container main body among the surfaces constituting the bottom surface portion. In the present specification, “downward” means a direction toward the bottom surface side of the sample liquid accommodation container, and “upward” means a direction toward the lid body side of the sample liquid accommodation container. In FIG. 2, “downward” means the direction of the arrow B.

Due to the inner surface of the bottom surface portion 12 having the deepest portion 12a and the inclined surface 12b inclined downward toward the deepest portion 12a, in a case where the level of the sample liquid has become extremely low, the sample liquid is gathered into the deepest portion 12a. The gathered sample liquid is sucked up with the use of the outflow channel 40, and thus the amount of sample liquid that remains unsucked can be reduced. Moreover, one end of the outflow channel 40 (second outflow channel 42) abuts against the bottom surface portion 12 (particularly, the deepest portion 12a), and this allows the sample liquid guided to the deepest portion 12a of the bottom surface portion 12 to be efficiently sucked up. Thus, the amount of sample liquid that remains unsucked can be further reduced. The configuration of the outflow channel 40 will be described in the following “(2-3) Other configurations”.

As illustrated in FIG. 2, the inclined surface 12b inclined downward toward the deepest portion 12a is preferably arranged to surround the deepest portion 12a. With this arrangement, the sample liquid remaining on the inner surface of the bottom surface portion 12 can be efficiently gathered into the deepest portion 12a. Furthermore, as illustrated in FIG. 2, the inclined surface 12b may be constituted by, for example, a surface having a uniform inclination angle.

As illustrated in FIG. 2, for example, the deepest portion 12a may have a planar shape, and, in particular, may have a planar and circular shape. Furthermore, the deepest portion 12a may have, for example, a point-like shape or a linear shape.

The inner surface of the bottom surface portion 12 illustrated in FIG. 2 is constituted by the deepest portion 12a and the inclined surface 12b inclined downward toward the deepest portion 12a. Specifically, the inner surface of the bottom surface portion 12 illustrated in FIG. 2 is constituted by the planar and circular deepest portion 12a and the inclined surface 12b inclined downward from a lower edge of the cylindrical body portion 13 toward the deepest portion 12a. However, the inner surface of the bottom surface portion 12 may include a surface other than the deepest portion 12a and the inclined surface 12b. For example, the inner surface of the bottom surface portion 12 may have a horizontal surface provided so as to protrude horizontally from the lower edge of the cylindrical body portion 13 toward the inside of the container main body 10. Furthermore, for example, the inner surface of the bottom surface portion 12 may have a second inclined surface that is inclined from the lower edge of the cylindrical body portion 13 toward the inside of the container main body 10 and has an inclination angle different from that of the inclined surface 12b. In these examples, the inclined surface 12b may be provided between the horizontal surface or the second inclined surface and the deepest portion 12a.

The material of the container main body 10 is preferably a material that does not deform with a change in internal pressure. The pressure inside the container main body 10 may change with inflow and outflow of the sample liquid. The container main body 10 is formed by a material that does not deform with a change in internal pressure, so that the sample liquid can be stably accommodated. Furthermore, the container main body 10 may be formed by, for example, a transparent material so that the state of the accommodated sample liquid is visible. Examples of a transparent material that does not deform with a change in pressure include a synthetic resin that has high rigidity and is transparent, and specific examples thereof include transparent ABS resin, polycarbonate (PC), and polyethylene (PE). For example, in a case where the lid body 20 is formed by soft PVC as described later, the container main body 10 may be formed by transparent ABS resin that can be welded to soft PVC.

The volume of the container main body 10 may be appropriately set by those skilled in the art, and may be, for example, 1 mL or more and 1000 mL or less. Note that the volume of the container main body 10 means the volume obtained by excluding the volume of a headspace (space that is not filled with the sample liquid) necessary for inflow and outflow of the sample liquid, from the fully filled volume of the container main body 10. That is, the volume of the container main body 10 means the maximum volume of the sample liquid that can flow in and out.

The outer diameter of the body portion 13 of the container main body 10 may be, for example, 35 mm or more, preferably 40 mm or more, more preferably 45 mm or more, and still more preferably 47 mm or more. With the outer diameter of the container main body 10 being within such a numerical range, the sample liquid is more efficiently stirred when the container main body 10 is rotationally shaken, and the microparticles in the sample liquid can be more favorably dispersed.

The inner diameter of the body portion 13 of the container main body 10 can be appropriately set by those skilled in the art. For example, in a case where the outer diameter of the body portion 13 of the container main body 10 is 47 mm or more, the inner diameter thereof may be 43 mm or more.

The length of the container main body 10 in the longitudinal direction may be appropriately set by those skilled in the art in accordance with the volume, the outer diameter, and the like of the container main body 10.

### (2-3) Other Configurations

Next, configurations other than that of the container main body 10 will be described with reference to FIG. 2. As described above, the lid body 20 has the top surface portion 21, and seals the opening 11 of the container main body 10. The top surface portion 21 may have any shape that allows for covering the circular opening 11, and may preferably have a circular shape. The lid body 20 may have, in addition to the top surface portion 21, a side surface portion 22 extending downward from the top surface portion 21. The side surface portion 22 may preferably be circular along the inner surface of the opening 11. With this arrangement, the opening 11 can be more reliably sealed.

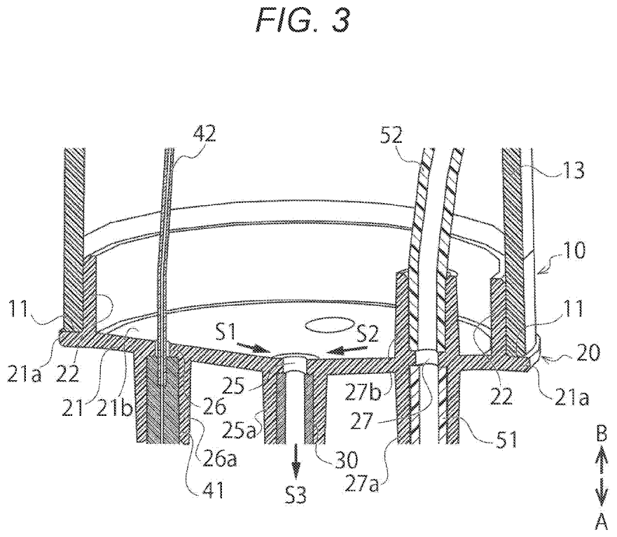

The lid body 20 will be described with reference to FIG. 3. FIG. 3 is a cross-sectional view illustrating the lid body 20 and the periphery thereof in a state where the sample liquid accommodation container 1 is turned upside down. In FIG. 3, the direction toward the bottom surface of the sample liquid accommodation container 1, that is, “downward” in the present specification, is the direction of the arrow B, and the direction toward the lid body of the sample liquid accommodation container 1, that is, “upward” in the present specification, is the direction of the arrow A.

As illustrated in FIG. 3, the top surface portion 21 of the lid body 20 may have a top surface edge 21a and the side surface portion 22. The top surface edge 21a is provided along the peripheral edge of the top surface portion 21. The side surface portion 22 extends downward from the inside of the top surface edge 21a, and is circular along the inner surface of the opening 11. The side surface portion 22 is fitted inside the opening 11 of the container main body 10. Furthermore, the top surface edge 21a is in contact with the upper edge of the opening 11 in a state where the side surface portion 22 is fitted inside the opening 11. As illustrated in FIG. 2, the opening 11 and the lid body 20 may be combined to seal the opening 11.

The opening 11 and the lid body 20 may be combined and then welded together. With this arrangement, sealability between the opening 11 and the lid body 20 can be improved. Thus, leakage of the sample liquid accommodated in the container main body 10 can be more reliably prevented.

Configurations other than that of the container main body 10 will be further described with reference to FIGS. 2 and 3. The top surface portion 21 of the lid body 20 has the first through hole 25. The vent pipe 30 may be connected to the first through hole 25. The vent pipe 30 is provided for the purpose of effectively eliminating a pressure difference caused by the sample liquid flowing into and out from the container main body 10. The first through hole 25 may have an outer cylindrical portion 25a extending from the peripheral edge thereof toward the outside of the container. The vent pipe 30 may be connected to the first through hole 25 by being fitted inside the outer cylindrical portion 25a.

The top surface portion 21 of the lid body 20 may have the second through hole 26. The second through hole 26 is connected with the outflow channel 40 for allowing the sample liquid to flow out from the container main body 10. That is, the outflow channel 40 is a pipe that communicates the inside and the outside of the sample liquid accommodation container 1 via the second through hole 26. As illustrated in FIG. 2, one end of the outflow channel 40 is located outside the sample liquid accommodation container 1, and the other one end of the outflow channel 40 is located inside the container main body 10. The other one end of the outflow channel 40 may preferably abut against the inner surface of the bottom surface portion 12, and more preferably abut against the inner surface of the deepest portion 12a of the bottom surface portion 12. With one end of the outflow channel 40 located inside the container main body 10 abutting against the inner surface of the bottom surface portion 12 (particularly, the deepest portion 12a), when the sample liquid in the container main body 10 is sucked up and flows out, the amount of sample liquid that remains unsucked can be reduced.

The outflow channel 40 is formed by one or a plurality of members. The outflow channel 40 may be formed by, for example, two members, that is, the first outflow channel 41 and the second outflow channel 42. The first outflow channel 41 may be arranged toward the outside of the sample liquid accommodation container 1, and the second outflow channel 42 may be arranged toward the inside of the sample liquid accommodation container 1.

The second through hole 26 may have the outer cylindrical portion 26a extending from the peripheral edge thereof toward the outside of the container. One end of the first outflow channel 41 may be fitted inside the outer cylindrical portion 26a. One end of the second outflow channel 42 may be inserted into the second through hole 26 (see FIG. 3). The other one end of the second outflow channel 42 may abut against the inner surface of the bottom surface portion 12 (particularly, the deepest portion 12a) of the container main body 10 (see FIG. 2). In this way, the first outflow channel 41 and the second outflow channel 42 may be connected to the second through hole 26. With this arrangement, the outflow channel 40 passing via the second through hole 26 may be formed.

The top surface portion 21 of the lid body 20 may have the third through hole 27. The third through hole 27 is connected with the inflow channel 50 for allowing the sample liquid to flow into the container main body 10. That is, the inflow channel 50 is a pipe that communicates the inside and the outside of the sample liquid accommodation container 1 via the third through hole 27. One end of the inflow channel 50 is located outside the sample liquid accommodation container 1. The other one end of the inflow channel 50 may be located inside the container main body 10, and preferably be located upward with respect to the liquid level of the sample liquid inside the container main body 10. A virtual line L in FIG. 2 indicates an example of the position of the liquid level. Since one end of the inflow channel 50 is located upward with respect to the liquid level of the sample liquid inside the container main body 10, it is possible to prevent the sample liquid in the container main body 10 from returning to the inflow channel 50 due to differential pressure after all the sample liquid has flown into the container main body 10 via the inflow channel 50. Note that the “liquid level of the sample liquid” described above is a liquid level of a maximum volume specified in advance. Specifically, in the microparticle sorting device using the sample liquid accommodation container 1, the maximum volume of the sample liquid accommodated in the sample liquid accommodation container 1 may be specified in advance. The liquid level when the sample liquid of the specified maximum solution is accommodated is the “liquid level of the sample liquid” described above.

The inflow channel 50 is formed by one or a plurality of members. The inflow channel 50 may be formed by, for example, two members, that is, a first inflow channel 51 and a second inflow channel 52. The first inflow channel 51 may be arranged toward the outside of the sample liquid accommodation container 1, and the second inflow channel 52 may be arranged toward the inside of the sample liquid accommodation container 1.

The third through hole 27 may have an outer cylindrical portion 27a extending from the peripheral edge thereof toward the outside of the container and an inner cylindrical portion 27b extending from the peripheral edge thereof toward the inside of the container. One end of the first inflow channel 51 may be fitted inside the outer cylindrical portion 27a. One end of the second inflow channel 52 may be fitted inside the inner cylindrical portion 27b (see FIG. 3). The other one end of the second inflow channel 52 may be located upward with respect to the liquid level of the sample liquid (see FIG. 2). In this way, the first inflow channel 51 and the second inflow channel 52 may be connected to the third through hole 27. With this arrangement, the inflow channel 50 passing via the third through hole 27 may be formed.

The vent pipe 30, the outflow channel 40 (first outflow channel 41 and second outflow channel 42), and the inflow channel 50 (first inflow channel 51 and second inflow channel 52) may be combined with the lid body 20 and then welded together. With this arrangement, it is possible to improve airtightness of the sample liquid accommodation container 1, and more reliably prevent leakage of the sample liquid accommodated in the container main body 10.

The structure of the inside of the lid body 20 will be described with reference to FIG. 3. The inner surface of the top surface portion 21 of the lid body 20 has an inclined surface 21b inclined upward with the first through hole 25 as the highest point. Thus, turning the sample liquid accommodation container 1 upside down allows the sample liquid to be gathered into the first through hole 25 and taken out from the first through hole 25. In a case where the sample liquid is present on the inclined surface 21b illustrated in FIG. 3, the sample liquid may move toward the through hole 25 as indicated by arrows S1 and S2, for example. Then, the sample liquid passes via the first through hole 25, passes through the inside of the vent pipe 30, and is drained to the outside of the lid body 20 as indicated by an arrow S3.

In the sample liquid accommodation container 1 of the present embodiment, the vent pipe 30 may be provided for the purpose of effectively eliminating a pressure difference caused by inflow and outflow of the sample liquid. However, when the sample liquid accommodation container 1 is turned upside down for the purpose of taking out the sample liquid, the vent pipe 30 may serve as a drain path through which the sample liquid passes. Since the vent pipe 30 also serves as a drain path, it is not necessary to separately provide means for taking out the sample liquid, and the configuration of the sample liquid accommodation container 1 can be simplified.

In the sample liquid accommodation container 1, the opening 11 is sealed by the lid body 20. Thus, the lid body 20 and the opening 11 are in close contact with each other, and it is difficult to remove the lid body 20 from the opening 11 in some cases. Furthermore, the lid body 20 and another component are welded together for improved sealability in some cases. In this case, it is difficult to separate the welded members and remove the lid body 20 from the opening 11. Thus, for example, when collecting residual sample liquid, it is difficult to remove the lid body 20 and take out the sample liquid in some cases. However, in the sample liquid accommodation container 1 of the present embodiment, the sample liquid can be taken out just by turning the sample liquid accommodation container 1 upside down as described above. That is, the inside of the top surface portion 21 of the lid body 20 has the inclined surface 21b described above, and this allows the sample liquid to be easily taken out.

As illustrated in FIG. 3, the inclined surface 21b inclined upward with the first through hole 25 as the highest point is preferably arranged to surround the first through hole 25. With this arrangement, when the sample liquid accommodation container 1 is turned upside down, the sample liquid on the inner surface of the top surface portion 21 is efficiently gathered into the first through hole 25. Thus, the sample liquid can be taken out more easily. Furthermore, as illustrated in FIG. 3, the inclined surface 21b may be constituted by, for example, a surface having a uniform inclination angle.

The inner surface of the top surface 21 of the lid body 20 may include a surface other than the inclined surface 21b inclined upward with the first through hole 25 as the highest point. For example, the inner surface of the top surface 21 may have a horizontal surface provided so as to protrude horizontally from the side surface portion 22 toward the inside of the container main body 10. Furthermore, for example, the inner surface of the top surface portion 21 may have a second inclined surface that is inclined from the side surface portion 22 toward the inside of the container main body 10 and has an inclination angle different from that of the inclined surface 21b. In these examples, the inclined surface 21b may be provided between the horizontal surface or the second inclined surface and the first through hole 25.

As described in “(2-2) Configuration of container main body” described above, the material of the lid body 20 is preferably a material that does not deform with a change in internal pressure. In a case where the lid body 20 is fitted inside the opening 11 to seal the opening 11, the material of the lid body 20 may be, for example, a material having flexibility. Furthermore, in a case where the lid body 20 and another member are welded together for improved sealability of the sample liquid accommodation container 1, the material of the lid body 20 may be, for example, a material having heat weldability. Examples of a material that does not deform with a change in internal pressure and has flexibility and heat weldability include soft polyvinyl chloride (soft PVC). By using soft PVC, the container main body 10 and the lid body 20 can be easily welded together.

The vent pipe 30, the outflow channel 40 (first outflow channel 41 and second outflow channel 42), and the inflow channel 50 (first inflow channel 51 and second inflow channel 52) may be, for example, tubes. The materials of the tubes may be appropriately selected by those skilled in the art. Furthermore, the cross-sectional dimensions of these channels may be appropriately set by those skilled in the art in accordance with the sample liquid that flows through the channels. For example, the materials of the tubes of the vent pipe 30, the first outflow channel, and the inflow channel 50 (first inflow channel 51 and second inflow channel 52) may be rubber such as butyl rubber, isoprene rubber, and natural rubber; a polymer elastomer such as a styrene-based elastomer, an olefin-based elastomer, a polyester-based elastomer, and a nylon-based elastomer; thermoplastic resin such as low-density polyethylene, high-density polyethylene, polypropylene, and cyclic polyolefin; or the like. Each of the tubes of the vent pipe 30, the first outflow channel, and the inflow channel 50 (first inflow channel 51 and second inflow channel 52) may be formed by a different material, or may be formed by the same material.

The tube of the second outflow channel 42 may be, for example, a PEEK tube that is 1/16 inch in outer diameter and 0.5 mm in inner diameter, or 1 mm in inner diameter.

### (2-4) Sample Liquid

A sample liquid that can be accommodated in the sample liquid accommodation container 1 of the present embodiment will be described. The sample liquid contains microparticles. Examples of the microparticles include bioparticles (biological microparticles) such as cells, cell masses, microorganisms, and ribosomes, or synthetic microparticles such as gel particles, beads, latex particles, polymer particles, and industrial particles.

The bioparticles described above may include chromosomes, ribosomes, mitochondria, and organelles (cell organelles) constituting various cells. The cells described above may include animal cells (e.g., blood cells) and plant cells. The cells may be particularly blood cells or tissue cells. The blood cells may be, for example, floating cells such as T cells or B cells. The tissue cells may be, for example, adherent cells separated from adherent cultured cells or tissues. The cell masses described above may include, for example, spheroids and organoids. The microorganisms described above may include bacteria such as Escherichia coli, viruses such as tobacco mosaic virus, and fungi such as yeast. Moreover, the bioparticles described above may also include biological polymers such as nucleic acids, proteins, and complexes thereof. These biological polymers may be, for example, those extracted from cells, or those contained in blood samples or other liquid samples.

The synthetic microparticles described above may be, for example, microparticles constituted by an organic or inorganic polymer material, metal, or the like. The organic polymer material may include polystyrene, a styrene/divinylbenzene copolymer, polymethyl methacrylate, and the like. The inorganic polymer material may include glass, silica, a magnetic material and the like. The metal may include gold colloid, aluminum, and the like. The synthetic microparticles may be, for example, gel particles or beads, and may be particularly gel particles or beads to which one or a combination of two or more selected from oligonucleotides, peptides, proteins, and enzymes are bound.

The shapes of the microparticles described above may be spherical or approximately spherical, or may be non-spherical. The size and mass of the microparticles may be appropriately selected by those skilled in the art. In the present technology, a chemical or biological label such as fluorescent dye or fluorescent protein, for example, may be attached to the microparticles as necessary. The label may make detection of the microparticles easier. The label to be attached can be appropriately selected by those skilled in the art. A molecule (e.g., antibody, aptamer, DNA, and RNA) that specifically reacts with the microparticles may be bound to the label.

In the present technology, the microparticles contained in the sample liquid may be preferably bioparticles, and more preferably cells.

### (3) Configuration of Microchip

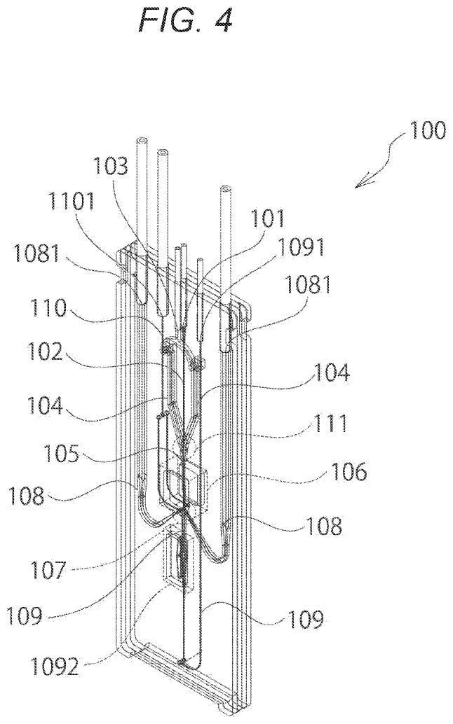

The configuration of the microchip 100 will be described with reference to FIG. 4. FIG. 4 is a schematic diagram illustrating an example of the microchip 100.

As illustrated in FIG. 4, the microchip 100 has a channel structure. In the microchip 100, a sample liquid inlet 101 and a terminal end 1091 of a sorting channel 109 are formed on the same side surface.

The microchip 100 is provided with the sample liquid inlet 101 into which a sample liquid is introduced, and a sheath liquid inlet 103 into which a sheath liquid is introduced. The sheath liquid inlet 103 is formed on the same side surface as the sample liquid inlet 101 and the terminal end 1091 of the sorting channel 109.

The sample liquid containing microparticles is introduced from the sample liquid inlet 101 into a sample liquid channel 102. The sheath liquid is introduced from the sheath liquid inlet 103 into a sheath liquid channel 104. The sheath liquid channel 104 branches into two sheath liquid channels 104 and 104. The two sheath liquid channels 104 and 104 pass through both sides of the sample liquid channel 102, and join with the sample liquid channel 102 at a joining portion 111. That is, each flow of the sheath liquid flowing through the sheath liquid channels 104 and 104 joins with the sample liquid flowing through the sample liquid channel 102 at the joining portion. With this arrangement, a laminar flow in which the sample liquid is surrounded by the sheath liquid is formed. The laminar flow flows through a main channel 105 toward a particle sorting unit 107.

The main channel 105 passes through an optical detection region 106. In the optical detection region 106, microparticles in the sample liquid are irradiated with light. Whether or not the microparticles are to be sorted can be determined on the basis of fluorescence and/or scattered light generated by the irradiation of light. In the particle sorting unit 107 in the microchip 100, the laminar flow described above that has flowed through the main channel 105 is separated and flows into two branch channels 108 and 108.

Note that the particle sorting unit 107 illustrated in FIG. 4 has the two branch channels 108 and 108, but the number of branch channels is not limited to two. That is, the particle sorting unit 107 may be provided with, for example, one or a plurality of (e.g., two, three, or four) branch channels.

A terminal end 1081 of the branch channel 108 is formed on the same side surface as the sample liquid inlet 101 and the terminal end 1091 of the sorting channel 109.

In the particle sorting unit 107, only in a case where microparticles (also referred to as “target sample”) to be sorted have flowed in, a flow entering the sorting channel 109 is formed, and the target sample is sorted. The flow entering the sorting channel 109 may be formed, for example, by generating a negative pressure in the sorting channel 109. In order to generate the negative pressure, for example, an excitation region 1092 may be provided, and an actuator or the like may be attached to the outside of the microchip 100 so that the wall of the excitation region 1092 can be deformed. The deformation of the wall of the excitation region 1092 may change an inner space of the excitation region 1092, thereby generating the negative pressure.

The actuator described above may be, for example, a piezo actuator. When the target sample is sucked into the sorting channel 109, the sample liquid and/or the sheath liquid may also flow into the sorting channel 109. In this way, the target sample may be sorted.

The main channel 105 and the sorting channel 109 communicate with each other via an orifice portion coaxial with the main channel 105. The target sample flows through the orifice portion into the sorting channel 109.

In order to prevent microparticles that are not to be sorted from entering the sorting channel 109 through the orifice portion described above, the orifice portion may include a buffer liquid channel 110. A buffer liquid is introduced into the buffer liquid channel 110 from a buffer liquid inlet 1101. A part of the introduced buffer liquid forms a flow from the orifice portion toward the main channel 105, so that microparticles that are not to be sorted may be prevented from entering the sorting channel 109.

The buffer liquid inlet 1101 is formed on the same side surface as the sample liquid inlet 101 and the terminal end 1091 of the sorting channel 109. Note that the rest of the introduced buffer liquid described above may flow into the sorting channel 109.

The laminar flow described above that has flowed into the branch channel 108 may be discharged to the outside of the microchip 100 at the terminal end 1081 of the branch channel 108. Furthermore, the microparticles sorted into the sorting channel 109 may be discharged to the outside of the microchip at the terminal end 1091 of the sorting channel. In this way, the target sample is sorted by the microchip 100.

Communication members may be inserted into the sample liquid inlet 101, the terminal end 1091 of the sorting channel 109, the sheath liquid inlet 103, the buffer liquid inlet 1101, and the terminal end 1081 of the branch channel 108. The microchip 100 may be coupled to other members included in the microparticle sorting kit 200 via the communication members.

The communication members described above include, for example, channels through which the sample liquid flows. The channels may be, for example, tubes. The materials of the tubes may be appropriately selected by those skilled in the art. The tubes may be, for example, polyvinyl chloride (PVC) tubes, silicone tubes, polyetheretherketone (PEEK) tubes, polytetrafluoroethylene (PTFE) tubes, or thermoplastic elastomer tubes, or a plurality of types of tubes may be coupled.

In the present technology, “micro” means that at least a part of a channel included in the microchip has a dimension on the order of μm, particularly a cross-sectional dimension on the order of μm. That is, in the present technology, the “microchip” refers to a chip including a channel on the order of μm, particularly a chip including a channel having a cross-sectional dimension on the order of μm. For example, a chip including a particle sorting unit constituted by a channel having a cross-sectional dimension on the order of μm may be referred to as a microchip according to the present technology.

The microchip 100 may be manufactured by a method known in the technical field. For example, the microchip 100 may be manufactured by bonding two or more substrates on which a predetermined channel is formed.

Examples of the material of the microchip 100 include polycarbonate, cycloolefin polymer, polypropylene, polydimethylsiloxane (PDMS), polymethyl methacrylate (PMMA), polyethylene, polystyrene, glass, and silicon.

### (4) Other Configurations

With reference to FIG. 1 again, configurations other than those of the sample liquid accommodation container 1 and the microchip 100 that can be included in the microparticle sorting kit 200 will be described.

The microparticle sorting kit 200 may include the pre-sample accommodation portion 2011 that accommodates the sample liquid and is located upstream of the sample liquid accommodation container 1. A substance that suppresses aggregation of microparticles in the sample liquid may be accommodated in the pre-sample accommodation portion 2011. The pre-sample accommodation portion 2011, and a waste portion 204, a sheath liquid accommodation portion 205, and a buffer liquid accommodation portion 206 described later may have, for example, a bag shape, and may be specifically plastic bags. Examples of the plastic bags may include polyethylene bags, polypropylene bags, polyvinyl chloride bags, or ethylene vinyl acetate copolymer bags.

As illustrated in FIG. 1, the microparticle sorting kit 200 may be provided with, for example, a filter portion 202 (202a and 202b). For example, the microparticle sorting kit 200 may include the pre-sample accommodation portion 2011, and may have the filter portion 202a between the pre-sample accommodation portion 2011 and the sample liquid accommodation container 1. Specifically, the filter portion 202a may be connected to the first inflow channel 51 of the sample liquid accommodation container 1, for example. With this arrangement, it is possible to prevent foreign matter from getting inside the sample liquid accommodation container 1. Furthermore, for example, the microparticle sorting kit 200 may have the filter portion 202b between the sample liquid accommodation container 1 and the microchip 100. In particular, the filter portion 202b may be arranged immediately before the microchip 100. With this arrangement, it is possible to prevent foreign matter from getting inside the microchip 100.

On the other hand, the microparticle sorting kit 200 need not have the filter portion 202a and/or the filter portion 202b described above. That is, the microparticle sorting kit 200 need not have the filter portion 202a between the pre-sample accommodation portion 2011 and the sample liquid accommodation container 1 and/or need not have the filter portion 202b between the sample liquid accommodation container 1 and the microchip 100. In the microparticle sorting kit 200, the sample liquid accommodation container 1 is detachable for the sake of simplification of the work to change the sample liquid and cost reduction. Depending on the application of the microparticle sorting kit 200, cost reduction may be more important than foreign matter contamination prevention. In this case, the filter portion 202a and/or the filter portion 202b need not be provided for the sake of further cost reduction.

The filter portion 202 (202a and 202b) may include, for example, a filter and a tapered portion located downstream of the filter. The sample liquid passes through the filter and then passes through the tapered portion. The tapered portion may prevent microparticles in the sample liquid from staying on an inner wall surface of the filter portion 202. Thus, the amount of microparticle loss can be reduced.

The filter portion 202 (202a and 202b) described above may further include a fitting portion as necessary. The fitting portion provides outer diameter fitting with a communication member for connection with the sample liquid accommodation container 1 and/or the microchip 100.

A target sample collection portion 203 can be continuously connected to the microchip 100. Specifically, the target sample collection portion 203 can be continuously connected to the terminal end 1091 (see FIG. 4) of the sorting channel 109 of the microchip 100. The target sample collection portion 203 accommodates sorted target microparticles.

The microparticle sorting kit 200 may be configured to allow the target sample collection portion 203 that accommodates the sorted target microparticles to continuously connect to the microchip 100 in a detachable manner. For example, the microparticle sorting kit 200 may be configured to allow the target sample collection portion 203 to continuously connect, in a detachable manner, to the microchip 100 (particularly, the terminal end 1091 of the sorting channel 109 of the microchip 100) via one or a plurality of communication members. In a state where the target sample collection portion 203 is continuously connected to the microchip 100 via one or a plurality of communication members, the sorted target microparticles can move from the microchip 100 to the target sample collection portion 203. The target sample collection portion 203 is removed from the microparticle sorting kit 200, for example, by separating the one or plurality of communication members from the target sample collection portion 203.

The microparticle sorting kit 200 may or need not include the target sample collection portion 203. That is, the target sample collection portion 203 may or need not belong to the set of components constituting the microparticle sorting kit 200. In a case where the microparticle sorting kit 200 does not include the target sample collection portion 203, for example, the user acquires, as necessary, the target sample collection portion 203 to be continuously connected to the microparticle sorting kit 200. That is, the target sample collection portion 203 may be prepared separately from the microparticle sorting kit 200 by the user.

The microparticle sorting kit 200 as described above allows the user to replace the target sample collection portion 203 whenever necessary. For example, when the user replaces the sample liquid accommodation container 1 in the microparticle sorting kit 200 in order to sort the target microparticles from a plurality of sample liquids, the user can also replace the target sample collection portion 203.

The target sample collection portion 203 may have, for example, a container shape, and specifically may have a bag shape (for example, a plastic bag) similar to the shape of the pre-sample accommodation portion 2011 described above or a container shape other than a bag shape. The target sample collection portion 203 is preferably a container other than a bag-shaped container, and more preferably a bottomed cylindrical container having one end as an opening and the other end as a bottom surface portion.

In the microchip 100 included in the microparticle sorting kit 200, when only the target sample is sorted from the sample liquid, microparticles that are not to be collected (hereinafter also referred to as “non-target sample”) need to be excluded. Furthermore, in the microchip 100, a sheath flow is used for sorting of the target sample, and it is therefore necessary to exclude a liquid containing the non-target sample, a so-called waste liquid. Thus, the microparticle sorting kit 200 may include, for example, the waste portion 204. The waste portion 204 is where the non-target sample can be discarded. The waste liquid portion 204 may include, for example, a communication member into which the waste liquid flows. The communication member may communicate with the terminal end 1081 (see FIG. 4) of the branch channel 108 of the microchip 100. With this arrangement, the target sample can be sorted and the non-target sample can be discarded.

Furthermore, in the microchip 100 included in the microparticle sorting kit, a sheath flow is formed. Thus, the microparticle sorting kit 200 may include, for example, the sheath liquid accommodation portion 205. The sheath liquid accommodation portion 205 accommodates a sheath liquid. The sheath liquid accommodation portion 205 may include, for example, a communication member into which the sheath liquid flows. The communication member may communicate with the sheath liquid inlet 103 (see FIG. 4) of the microchip 100. With this arrangement, the sheath liquid flows into the sheath liquid channel 104 of the microchip 100, and the sheath flow is formed. In order to drain the sheath liquid from the sheath liquid accommodation portion 205, for example, a drive source such as an actuator may be used.

The buffer liquid accommodation portion 206 accommodates a buffer liquid. The buffer liquid accommodation portion 206 may include, for example, a communication member into which the buffer liquid flows. The communication member may communicate with the buffer liquid inlet 1101 (see FIG. 4) of the microchip 100. With this arrangement, the buffer liquid flows into a channel in the microchip 100, and the target sample can be sorted. In order to drain the buffer liquid from the buffer liquid accommodation portion 206, for example, a drive source such as an actuator may be used.

When a part or all of a liquid in the microparticle sorting kit 200 is fed by a pump, fluctuation in flow rate (e.g., pulsation) caused by the pump may make an influence on the flow rate in the microchip 100 (particularly, the flow rate in the sorting channel 109) and sorting of microparticles in the particle sorting unit 107. In order to reduce the influence, the liquid feeding by the pump is desirably performed at a pressure as constant as possible. In order to keep the pressure by the liquid feeding as constant as possible, the microparticle sorting kit 200 may include dampers 207 and a pressure sensor 208 that gauges the pressure for each of the dampers 207. With this arrangement, liquid feeding can be stably performed. The dampers 207 and the pressure gauge sensor 208 may in particular be arranged in the channel between the sheath liquid accommodation portion 205 and the microchip and/or in the channel between the buffer liquid accommodation portion 206 and the microchip 100.

As illustrated in FIG. 1, the microparticle sorting kit 200 may further include a sample liquid feeding mechanism 305. The sample liquid feeding mechanism 305 may be arranged in the channel between the sample liquid accommodation container 1 and the microchip 100.

The sample liquid feeding mechanism 305 may be, for example, a pump. The pump may be, for example, a peristaltic pump (tube pump), a roller pump, a syringe pump, or a centrifugal pump. The pump may in particular be a peristaltic pump or a roller pump for more precise control of the flow rate.

The microparticle sorting kit 200 may include a plurality of the sample liquid feeding mechanisms 305 as necessary. For example, the sample liquid feeding mechanisms 305 may be arranged in one or a plurality of channels selected from the channel between the microchip 100 and the waste portion 204, the channel between the sheath liquid accommodation portion 205 and the microchip 100, and the channel between the buffer liquid accommodation portion 206 and the microchip 100.

In the microparticle sorting kit 200, portions other than portions where the detachable components are continuously connected may communicate with each other by means of sealed connection.

The microparticle sorting kit 200 may further include, for example, a microchip located downstream of the target sample collection portion 203. With this arrangement, the target sample sorted from the sample liquid may be further sorted.

In the microparticle sorting kit 200, the number of each component may be one or more.

### (5) Modifications

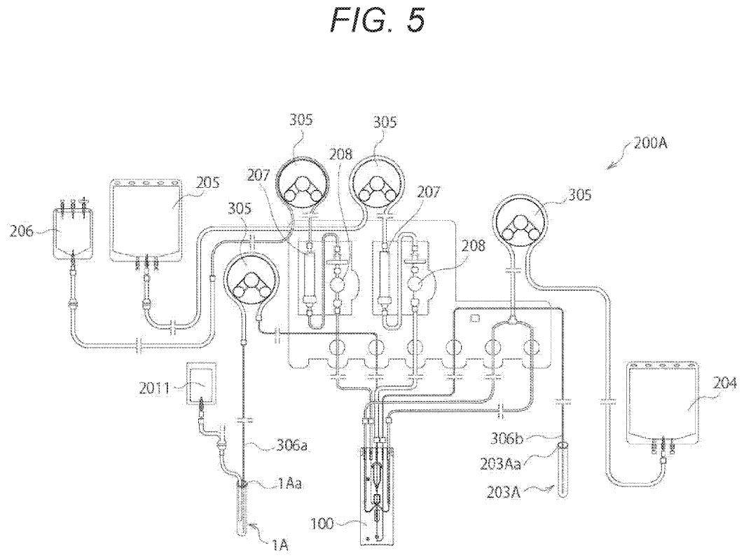

An overall configuration of a microparticle sorting kit 200A according to a modification of the first embodiment will be described with reference to FIG. 5. FIG. 5 is a schematic diagram illustrating an example of the microparticle sorting kit 200A according to the modification of the first embodiment. Hereinafter, the present modification will be described focusing on differences from the microparticle sorting kit 200 according to the first embodiment.

The microparticle sorting kit 200A includes a sample liquid accommodation container 1A instead of the sample liquid accommodation container 1 (FIG. 2) of the microparticle sorting kit 200. The microparticle sorting kit 200A is configured to allow the sample liquid accommodation container 1A to continuously connect to the microchip 100 in a detachable manner.

The sample liquid accommodation container 1A is a bottomed cylindrical container having one end as an opening 1Aa and the other end as a bottom surface portion. The sample liquid accommodation container 1A is continuously connected, in a detachable manner, to the microchip 100 via a communication member 306a that can be inserted and removed through the opening 1Aa.

As illustrated in FIG. 5, the sample liquid accommodation container 1A is preferably not provided with a member that closes the opening 1Aa such as a lid body. That is, the opening 1Aa is preferably not closed. With this arrangement, it is possible to insert and remove the communication member 306a and replace the sample liquid accommodation container 1A in a simple manner, so that the work to replace the sample liquid can be further simplified.

Furthermore, the microparticle sorting kit 200A includes a target sample collection portion 203A instead of the target sample collection portion 203 (FIG. 1) of the microparticle sorting kit 200. The microparticle sorting kit 200A is configured to allow the target sample collection portion 203A that accommodates sorted target microparticles to continuously connect to the microchip 100 in a detachable manner.

The target sample collection portion 203A is a bottomed cylindrical container having one end as an opening 203Aa and the other end as a bottom surface portion. The target sample collection portion 203A is continuously connected, in a detachable manner, to the microchip 100 via a communication member 306b that can be inserted and removed through the opening 203Aa.

As illustrated in FIG. 5, the target sample collection portion 203A is preferably not provided with a member that closes the opening 203Aa such as a lid body. That is, the opening 203Aa is preferably not closed. With this arrangement, it is possible to insertion and remove the communication member 306b in a simple manner, so that the work to replace the target sample collection portion 203A can be further simplified.

As opposed to the microparticle sorting kit 200 (FIG. 1), the microparticle sorting kit 200A does not include the filter portion 202 (202a and 202b). That is, the microparticle sorting kit 200A does not include the filter portion 202a between the pre-sample accommodation portion 2011 and the sample liquid accommodation container 1A, and does not include the filter portion 202b between the sample liquid accommodation container 1 and the microchip 100. Since the filter portion 202 (202a and 202b) is not included, the cost can be reduced.

### 2. SECOND EMBODIMENT (MICROPARTICLE SORTING DEVICE)

### (1) Overall Configuration

A microparticle sorting device according to a second embodiment of the present technology will be described. The microparticle sorting device according to the second embodiment is used with a microparticle sorting kit attached to the microparticle sorting device. The microparticle sorting kit is preferably, but not limited to, the microparticle sorting kit according to the first embodiment. Hereinafter, the microparticle sorting device according to the present embodiment will be described on the basis of an example where the microparticle sorting kit according to the first embodiment is attached to the microparticle sorting device. Therefore, the description regarding the microparticle sorting kit in the above 1. also applies to the present embodiment.

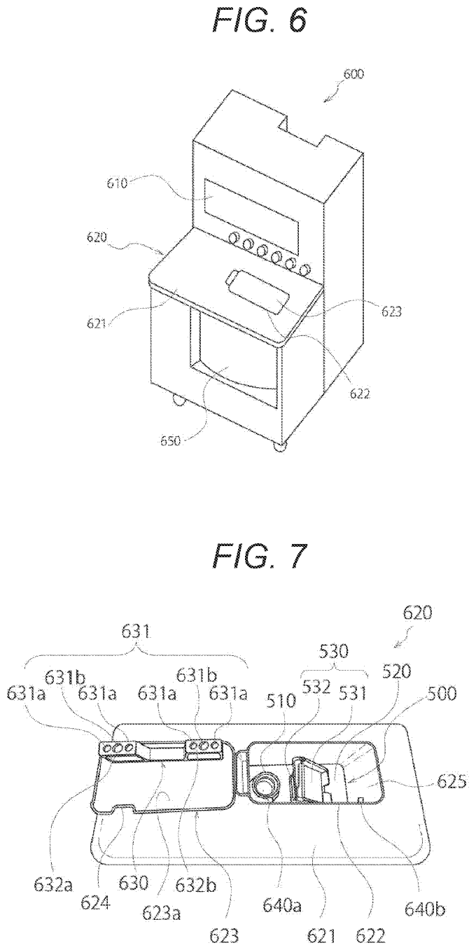

An overall configuration of a microparticle sorting device 600 according to the second embodiment will be described with reference to FIG. 6. FIG. 6 is a schematic diagram illustrating an example of the microparticle sorting device 600 according to the second embodiment.

The microparticle sorting device 600 includes a kit attachment surface 610 to which the microparticle sorting kit is detachably attached. The kit attachment surface 610 can be arranged along the vertical direction at an upper part of the microparticle sorting device 600.

The microparticle sorting device 600 includes a top plate portion 621 having a top plate opening 622 and a lid plate portion 623 capable of opening and closing the top plate opening 622, and further includes a storage portion 620 having a space therein. The top plate portion 621 can be arranged along the horizontal direction at a middle part of the microparticle sorting device 600.

The microparticle sorting device 600 includes a first holding portion capable of holding a sample liquid accommodation container that accommodates a sample liquid containing microparticles. The first holding portion is provided in the space of the storage portion 620. The sample liquid accommodation container may be as described in the above 1., and the description also applies to the sample liquid accommodation container held by the first holding portion.

The microparticle sorting device 600 includes a second holding portion capable of holding a container. The second holding portion is provided on a first surface of the lid plate portion 623. The lid plate portion 623 can close the top plate opening 622 with the first surface and the second holding portion facing the space of the storage portion.

The microparticle sorting device 600 may include, for example, an accommodation portion 650 having an accommodation space therein. For example, the sheath liquid accommodation portion 205 illustrated in FIGS. 1 and 5 can be accommodated in the accommodation portion 650.

### (2) Configuration of Storage Portion

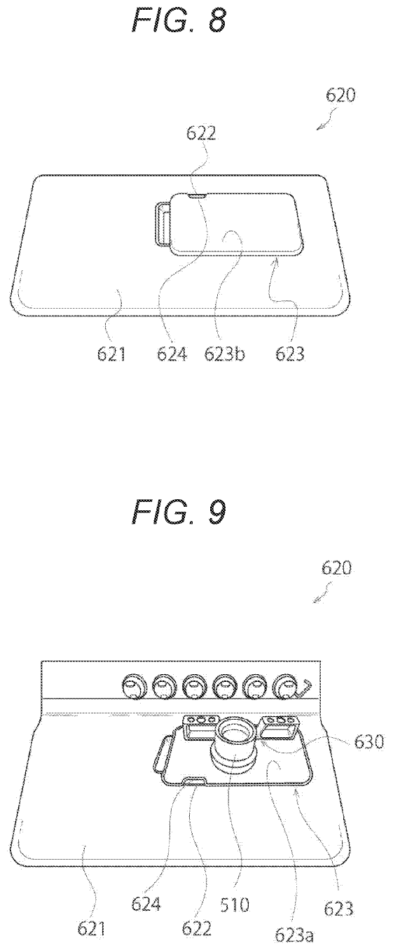

The configuration of the storage portion 620 will be described with reference to FIGS. 7 to 9. FIG. 7 is a schematic diagram illustrating a part of the storage portion 620 with the top plate opening 622 in an open state. FIG. 8 is a schematic diagram illustrating a part of the storage portion 620 with the top plate opening 622 in a closed state. FIG. 9 is a schematic diagram illustrating the lid plate portion 623, a first holding portion 510, and a second holding portion 630.

As illustrated in FIG. 7, the storage portion 620 has a space 625 therein. A temperature of the space 625 of the storage portion 620 may be controllable, and may be adjusted in accordance with, for example, the type of microparticles to be sorted and sorting conditions.

As illustrated in FIG. 8, the storage portion 620 can be a closed space by the lid plate portion 623 bringing the top plate opening 622 into a closed state. With this arrangement, the space 625 (FIG. 7) of the storage portion 620 is easily maintained at the adjusted temperature. Furthermore, in a case where the lid plate portion 623 includes a light-shielding material, so that it is possible to prevent light from entering the space 625.

As illustrated in FIG. 7, the first holding portion 510 capable of holding the sample liquid accommodation container is provided in the space 625 of the storage portion 620. It is preferable that the first holding portion 510 be provided in the space 625 of the storage portion 620 in a detachable manner. For example, as illustrated in FIG. 9, the first holding portion 510 can be used outside the space of the storage portion 620. Therefore, the user can easily cause the first holding portion 510 to hold the sample liquid accommodation container, and can easily remove the sample liquid accommodation container from the first holding portion 510. Furthermore, when the user performs work using the sample liquid accommodation container (for example, when the user performs work to take out the sample liquid from the sample liquid accommodation container), the user can use the first holding portion 510 at any place as a space where the sample liquid accommodation container is placed.

As illustrated in FIG. 7, in the space 625 of the storage portion 620, for example, a sample liquid stirring device 500 for stirring the sample liquid accommodated in the sample liquid accommodation container may be provided. That is, the microparticle sorting device 600 may further include the sample liquid stirring device 500 located in the space 625 of the storage portion 620. The sample liquid stirring device 500 is a device that stirs the sample liquid by rotationally shaking the sample liquid accommodation container. The sample liquid stirring device 500 can favorably disperse microparticles in the sample liquid accommodated in the sample liquid accommodation container by performing rotational shaking type stirring.

In a case where the microparticle sorting device 600 includes the sample liquid stirring device 500, the first holding portion 510 may be provided in the sample liquid stirring device 500, and may be preferably provided in a detachable manner. The first holding portion 510 attached to the sample liquid stirring device 500 can prevent the sample liquid accommodation container from falling over at the time of rotational shaking. Furthermore, since the first holding portion 510 can be used outside the space 625 of the storage portion 620, there is an advantage that the first holding portion 510 can be used at any place as described above.

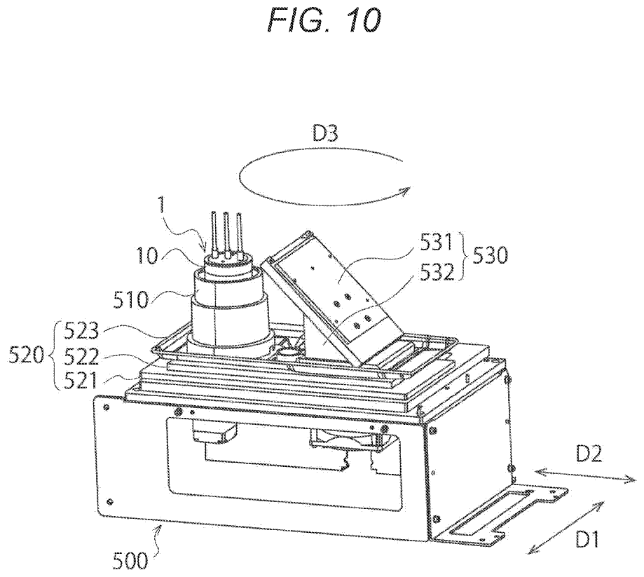

The sample liquid stirring device 500 will be described with reference to FIG. 10. FIG. 10 is a schematic diagram illustrating an example of the sample liquid stirring device 500. Note that FIG. 7 described above illustrates, for the sake of simplicity, only an extracted part of the sample liquid stirring device 500 illustrated in FIG. 10.

As illustrated in FIG. 10, the sample liquid stirring device 500 includes the first holding portion 510 and a rotation portion 520 that causes the first holding portion 510 to perform a horizontal circular motion. Hereinafter, a case where the sample liquid accommodation container 1 described in the above 1. is held by the first holding portion 510 will be described as an example. The sample liquid accommodation container used in the microparticle sorting device 600, however, is not limited to the sample liquid accommodation container 1.

The first holding portion 510 preferably fixes the sample liquid accommodation container 1 in such a way that the radial direction of the sample liquid accommodation container 1 is horizontal. The “fixes the sample liquid accommodation container 1 in such a way that the radial direction of the sample liquid accommodation container 1 is horizontal” means fixing the sample liquid accommodation container 1 so that the longitudinal direction of the sample liquid accommodation container 1 is perpendicular to the surface on which the sample liquid stirring device 500 is installed.

The first holding portion 510 may have, for example, a cylindrical cavity therein. The sample liquid accommodation container 1 can be arranged in the cavity. The inner diameter of the cavity may be, for example, approximately equivalent to the outer diameter of the container main body 10 of the sample liquid accommodation container 1. The depth of the cavity may be, for example, 50% or more, 60% or more, or 70% or more of the length in the longitudinal direction of the container main body 10 of the sample liquid accommodation container 1. With the cavity having such an inner diameter and depth, the sample liquid accommodation container 1 can be more stably fixed.

The rotation portion 520 may have, for example, a first plate portion 521, a second plate portion 522, and a third plate portion 523 in this order from the bottom. The first holding portion 510 may be arranged on the third plate portion 523. The first plate portion 521 may perform a reciprocating linear motion in a first horizontal direction (e.g., the direction of an arrow D1 illustrated in FIG. 10), for example. The second plate portion 522 may perform a reciprocating linear motion in a second horizontal direction (e.g., the direction of an arrow D2 illustrated in FIG. 10) orthogonal to the first horizontal direction, for example. For example, the third plate portion 523 may perform a horizontal circular motion by a movement obtained by combining these reciprocating linear motions that are horizontally orthogonal to each other. As a result, the first holding portion 510 arranged on the third plate portion 523 may perform a horizontal circular motion. In this way, the rotation portion 520 may cause the first holding portion 510 to perform a horizontal circular motion. An arrow D3 in FIG. 10 shows an example of the direction of the circular motion of the rotation portion 520 (third plate portion 523) and the first holding portion 510.

A power source for causing the rotation portion 520 to perform a horizontal circular motion may be, for example, a motor. The type of the motor may be appropriately selected by those skilled in the art.

As described above, in the sample liquid stirring device 500 of the present embodiment, the first holding portion 510 performs a horizontal circular motion. With this arrangement, the sample liquid stirring device 500 can rotationally shake the sample liquid accommodation container 1 fixed to the first holding portion 510. By the rotational shaking, the sample liquid in the sample liquid accommodation container 1 is effectively stirred and mixed well. Thus, the sample liquid stirring device 500 can favorably disperse the microparticles in the sample liquid to make the concentration of the sample liquid in the sample liquid accommodation container 1 uniform. As a result, the sample liquid stirring device 500 can make the concentration of the sample liquid fed from the sample liquid accommodation container 1 almost constant.

The rotation portion 520 may cause the first holding portion 510 to perform a horizontal circular motion either in a continuous manner or in an intermittent manner. That is, the horizontal circular motion may be a continuous motion, or may be an intermittent motion. The intermittent motion is to repeat motion and stop. For example, the intermittent motion may be selected in a case where the concentration of the sample liquid fed from the sample liquid accommodation container 1 is almost constant when the horizontal circular motion is the intermittent motion. With this arrangement, a load applied to the power source (e.g., a motor) for causing the rotation portion 520 to perform a horizontal circular motion can be reduced. For example, in a case where the intermittent motion results in an increase in fluctuation of the concentration of the sample liquid that is fed, the continuous motion may be selected. As described above, either the continuous motion or the intermittent motion may be selected for the horizontal circular motion depending on the fluctuation of the concentration of the sample liquid fed from the sample liquid accommodation container 1.

The sample liquid stirring device 500 may further include a stand portion 530. The stand portion 530 may have, for example, an inclined plate portion 531 inclined relative to the horizontal direction and a support portion 532 that supports the inclined plate portion 531. The stand portion 530 may be disposed on, for example, the third plate portion 523 of the rotation portion 520.

As described in the above 1., the target microparticles sorted from the sample liquid can be accommodated in, for example, the target sample collection portion. The stand portion 530 in the sample liquid stirring device 500 may be, for example, a stand on which the target sample collection portion is placed.

The configuration of the storage portion 620 will be further described with reference to FIGS. 7 to 9 again. The storage portion 620 includes the top plate portion 621, the top plate opening 622 provided in the top plate portion 621, and the lid plate portion 623 having a first surface 623a and capable of opening and closing the top plate opening 622. In the present specification, the “lid plate portion capable of opening and closing the top plate opening” refers to a lid plate portion that can bring the top plate opening into an open state or a closed state. The “the top plate opening in a closed state” includes, but is not limited to, a state where the top plate opening is fully closed, and may include, for example, a state where the top plate opening is partially closed (a state where the top plate opening is partially opened). That is, the lid plate portion may bring the top plate opening portion into a fully or partially closed state. FIGS. 7 to 9 illustrate an example of the lid plate portion that brings the top plate opening into a partially closed state (partially open state). Specifically, FIGS. 7 to 9 illustrate the lid plate portion 623 having a cutout portion 624. As illustrated in FIG. 8, a part of the top plate opening 622 (a portion corresponding to the cutout portion 624) is in an open state.

The lid plate portion 623 has the first surface 623a (FIG. 7) and a second surface 623b (FIG. 8) on an opposite side from the first surface 623a. As illustrated in FIG. 7, the second holding portion 630 capable of holding a container is provided on the first surface 623a of the lid plate portion 623. The second holding portion 630 can be used in a state where the first surface 623a of the lid plate portion 623 faces upward. That is, the second holding portion 630 can be used in a state where the lid plate portion 623 is placed with the second surface 623b facing downward.

In a state where the lid plate portion 623 has brought the top plate opening 622 into a closed state (FIG. 8), the first surface 623a of the lid plate portion 623 and the second holding portion 630 are positioned facing the space 625 of the storage portion 620, and the second surface 623b of the lid plate portion 623 is positioned facing outward. As described above, the lid plate portion 623 can close the top plate opening 622 with the first surface 623a and the second holding portion 630 facing the space 625 of the storage portion 620.

As an example, the lid plate portion 623 is connected to the top plate opening 622 by a hinge portion, and may be pivotable about the hinge portion. The user can turn the lid plate portion 623 to bring the top plate opening 622 in an open state, and can place the second surface 623b of the lid plate portion 623 on the top plate portion 621 to cause the first surface 623a of the lid plate portion 623 to face upward. With this arrangement, the lid plate portion 623 is brought into a state as illustrated in FIG. 7, that is, the second holding portion 630 is made available for use.

As another example, the lid plate portion 623 may be separable from the top plate opening 622. In the present specification, “separable from the top plate opening” means that it can be completely separated from the top plate opening. Since the lid plate portion 623 is separable from the top plate opening 622, the user can move the lid plate portion 623 to any position and can use the second holding portion 630 at any place. For example, the user can separate the lid plate portion 623 closing the top plate opening 622 (FIG. 8) from the top plate opening 622 and invert the lid plate portion 623 to cause the first surface 623a to face upward, and then place the lid plate portion 623 on the top plate portion 621 to close the top plate opening 622 as illustrated in FIG. 9.

It is only required that the second holding portion 630 be capable of holding one or a plurality of containers, the second holding portion 630 is preferably capable of holding a plurality of containers, and more preferably capable of holding a plurality of containers having different capacities. The second holding portion 630 illustrated in FIG. 7 is capable of holding a plurality of containers having different capacities. Specifically, the second holding portion 630 illustrated in FIG. 7 has a plurality of holding holes 631 into which containers can be inserted, and the plurality of holding holes 631 includes holding holes 631a and 631b having different hole diameters. For example, the holding holes 631a and 631b can hold a plurality of cylindrical containers having different outer diameters.

The second holding portion 630 may be capable of holding, for example, a sample liquid accommodation container and/or a target sample collection portion. The sample liquid accommodation container and the target sample collection portion held by the second holding portion 630 are preferably containers other than bag-shaped containers, and more preferably bottomed cylindrical containers having one end as an opening and the other end as a bottom surface portion.

In a case of changing the sample liquid by replacing the sample liquid accommodation container as described in the above 1., the second holding portion 630 can contribute to simplification of the work to change the sample liquid and improvement of user convenience. For example, the use of the second holding portion 630 allows a replacement sample liquid accommodation container to be placed near the sample liquid accommodation container held by the first holding portion 510. Furthermore, for example, the use of the second holding portion 630 capable of holding a plurality of containers without using the first holding portion 510 allows a plurality of replacement sample liquid accommodation containers to be placed side by side on the second holding portion 630. It is possible to perform, by placing the plurality of sample liquid accommodation containers close to each other as in these examples, the work to replace the sample liquid accommodation container in a simple and quick manner. Furthermore, it is possible to save, by using the second holding portion 630, time and effort to prepare a separate member for holding a replacement sample liquid accommodation container.

As illustrated in FIG. 7, the microparticle sorting device 600 may further include an opening and closing detector 640 (640a and 640b) capable of detecting whether or not the lid plate portion 623 closes the top plate opening 622. Although FIG. 7 illustrates two opening and closing detectors 640a and 640b, the number of opening and closing detectors 640 is not limited to two, and may be one or more. As illustrated in FIG. 7, the opening and closing detector 640 (640a and 640b) may be provided inside or near the top plate opening 622. For example, the opening and closing detector 640 (640a and 640b) may be capable of detecting that the lid plate portion 623 closes the top plate opening 622 in a case where the opening and closing detector 640 is in contact with the first surface 623a of the lid plate portion 623 or the second holding portion 630. In this case, the opening and closing detector 640 (640a and 640b) can be configured to come into contact with the first surface 623a or the second holding portion 630 in a state where the lid plate portion 623 closes the top plate opening 622 with the first surface 623a and the second holding portion 630 facing the space 625 of the storage portion 620. As an example, the second holding portion 630 illustrated in FIG. 7 includes holding stand portions 632a and 632b, and the holding stand portions 632a and 632b are able to come into contact with the opening and closing detectors 640a and 640b. Specifically, in a state where the lid plate portion 623 closes the top plate opening 622 with the first surface 623a and the second holding portion 630 facing the space 625 of the storage portion 620, the holding stand portion 632a is in contact with the opening and closing detector 640a, and the holding stand portion 632b is in contact with the opening and closing detection portion 640b.

### (3) Other Configurations

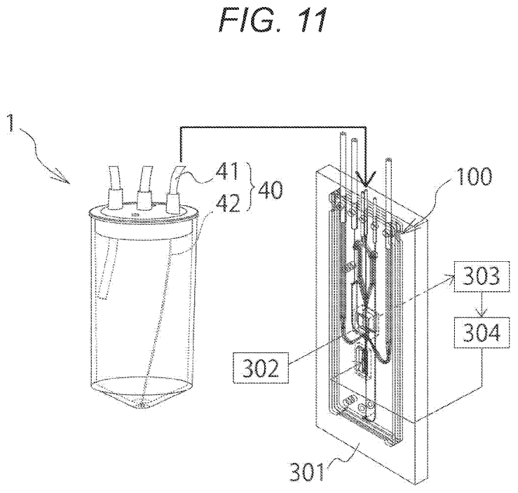

Configurations other than the above that can be included in the microparticle sorting device according to the present embodiment will be described with reference to FIG. 11. FIG. 11 is a schematic diagram illustrating some of the configurations included in the microparticle sorting device. FIG. 11 illustrates, as an example, the sample liquid accommodation container 1 described in the above 1. The sample liquid accommodation container used in the microparticle sorting device according to the present embodiment, however, is not limited to the sample liquid accommodation container 1.

The microparticle sorting device may further include a chip insertion unit 301 into which the microchip 100 is inserted, a light irradiation unit 302 that irradiates microparticles flowing through the main channel with light, a light detection unit 303 that detects scattered light and/or fluorescence emitted from the microparticles, and a control unit 304 that controls a traveling direction of the microparticles flowing through the main channel on the basis of data detected by the light detection unit 303. The chip insertion unit 301, the light irradiation unit 302, the light detection unit 303, and the control unit 304 will be further described below.

The chip insertion unit 301 has a structure into which the microchip 100 is inserted. The structure may be appropriately selected by those skilled in the art.

The light irradiation unit 302 irradiates, with light (e.g., excitation light), microparticles flowing through the main channel 105 (see FIG. 4) passing through the optical detection region 106. The light irradiation unit 302 may include, for example, a light source and an objective lens. The light source emits light toward the main channel. The light source may be appropriately selected by those skilled in the art in accordance with the purpose of sorting or the like, and may be, for example, a laser diode, an SHG laser, a solid-state laser, a gas laser, or a high-intensity LED, or may be a combination of two or more thereof. The objective lens gathers light (e.g., excitation light) for the microparticles flowing through the optical detection region 106.

The light irradiation unit 302 may further include another optical element as necessary. For example, the light irradiation unit 302 may irradiate one position in the optical detection region 106 with light, or may irradiate each one of a plurality of positions with light. For example, the light irradiation unit 302 may irradiate each of two different positions in the optical detection region 106 with light.

The light detection unit 303 detects scattered light and/or fluorescence emitted from the microparticles by light irradiation by the light irradiation unit 302. The light detection unit 303 may include, for example, a condenser lens and a detector. The condenser lens gathers scattered light and/or fluorescence emitted from the microparticles. The detector may be, for example, a PMT, a photodiode, a CCD, or a CMOS.

The light detection unit 303 may further include another optical element as necessary. The light detection unit 303 may further include, for example, a spectroscopic unit. Examples of an optical component constituting the spectroscopic unit may include a grating, a prism, and an optical filter. Including the spectroscopic unit allows the light detection unit 303 to detect light having a wavelength to be detected, separately from light having other wavelengths, for example.

The fluorescence detected by the light detection unit 303 may be, but is not limited to, fluorescence emitted from the microparticles themselves and fluorescence emitted from a substance (e.g., a fluorescent substance) with which the microparticles are labeled. The scattered light detected by the light detection unit 303 may be forward scattered light, side scattered light, Rayleigh scattering, Mie scattering, or a combination thereof.

The control unit 304 controls the traveling direction of the microparticles flowing through the main channel 105 on the basis of data (e.g., information regarding light) detected by the light detection unit 303. For example, the control unit 304 controls sorting of the microparticles on the basis of the data. For example, in a case where light detected by the light detection unit 303 satisfies a predetermined standard, the control unit 304 may determine to sort the microparticles. From light (scattered light and/or fluorescence) detected by the light detection unit 303, information regarding the light may be generated. The information may be generated, for example, by converting the light into an electric signal. In order to generate the information, the microparticle sorting device 300 may include an information generation unit configured to generate, from light detected by the light detection unit 303, information regarding the light. The information generation unit may be included in the control unit 304, or may be provided in the microparticle sorting device 300 as a component different from the control unit 304 without being included in the control unit 304. The control unit 304 may determine whether or not the light detected by the light detection unit 303 satisfies a predetermined standard on the basis of the information regarding the light. The control unit 304 may control sorting of the microparticles on the basis of a result of the determination.

In a case where it is determined that the microparticles are to be sorted on the basis of the result of the determination described above, the control unit 304 may change the flow in the channel so that the microparticles travels to the sorting channel 109 (see FIG. 4) through an orifice. The flow may be changed, for example, by decreasing the pressure in the sorting channel 109. Furthermore, after the microparticles haves been sorted, the control unit 304 may change the flow in the channel again. The flow may be changed again by increasing the pressure in the sorting channel 109. That is, the control unit 304 may control the pressure in the sorting channel 109 on the basis of the information regarding the light detected by the light detection unit 303.

The control unit 304 may have a function similar to that of the drive unit described in Japanese Patent Application Laid-Open No. 2014-036604, for example. That is, the control unit 304 can control an actuator configured to generate a negative pressure in the sorting channel 109. In a case where it is determined that the microparticles are to be sorted on the basis of the information regarding the light described above, the control unit 304 drives the actuator to generate a negative pressure in the sorting channel 109. With this arrangement, the microparticles to be sorted are sorted into the sorting channel 109. In a case where it is determined that the microparticles are not be sorted on the basis of the information regarding the light described above, the control unit 304 does not drive the actuator. Thus, the microparticles that are not to be sorted flows into the branch channel 108.

The actuator described above may be, for example, a piezoelectric element such as a piezo element. In a case where it is determined that the microparticles are to be sorted, the control unit 304 applies a voltage that causes piezoelectric contraction to the piezo element, to increase the volume in the sorting channel 109. As the volume increases, a negative pressure is generated in the sorting channel 109. With this arrangement, a flow from the main channel 105 to the sorting channel 109 is formed, and the microparticles are sorted into the sorting channel 109. In a case where it is determined that the microparticles are not to be sorted, the control unit 304 does not apply a voltage to the piezo element. Thus, a flow into the sorting channel 109 is not formed, and the microparticles flow into the branch channel 108.

The present technology may also take the following configuration.

[1]

A microparticle sorting device including:
a kit attachment surface to which a microparticle sorting kit is detachably attached;
a storage portion including a top plate portion having a top plate opening and a lid plate portion having a first surface and capable of opening and closing the top plate opening, the storage portion having a space therein;
a first holding portion capable of holding a sample liquid accommodation container configured to accommodate a sample liquid containing microparticles; and
a second holding portion capable of holding a container, in which
the first holding portion is provided in the space of the storage portion,
the second holding portion is provided on the first surface of the lid plate portion, and
the lid plate portion is capable of closing the top plate opening with the first surface and the second holding portion facing the space of the storage portion.
   [2]

The microparticle sorting device according to [1], in which the lid plate portion is separable from the top plate opening.

[3]

The microparticle sorting device according to [1] or [2], in which the first holding portion is detachably provided in the space of the storage portion.

[4]

The microparticle sorting device according to any one of [1] to [3], further including
a sample liquid stirring device located in the space of the storage portion, in which
the first holding portion is provided in the sample liquid stirring device, and
the sample liquid stirring device includes a rotation portion configured to cause the first holding portion to perform a horizontal circular motion.
   [5]

The microparticle sorting device according to [4], in which the first holding portion is detachably provided in the sample liquid stirring device.

[6]

The microparticle sorting device according to any one [1] to [5], in which the second holding portion is capable of holding a plurality of the containers.

[7]

The microparticle sorting device according to any one [1] to [5], in which the second holding portion is capable of holding a plurality of the containers having different capacities.

[8]

The microparticle sorting device according to any one of [1] to [7], in which
the microparticle sorting kit includes a microchip, the microchip including: a sample liquid inlet into which the sample liquid containing the microparticles accommodated in the sample liquid accommodation container is introduced; a main channel through which the sample liquid that has been introduced flows; and a sorting channel where target microparticles are sorted from the sample liquid flowing through the main channel.
   [9]

The microparticle sorting device according to [8], in which the microparticle sorting kit is configured to allow the sample liquid accommodation container to continuously connect to the microchip in a detachable manner.

[10]

The microparticle sorting device according to [8] or [9], in which the microparticle sorting kit is configured to allow a target sample collection portion to continuously connect to the microchip in a detachable manner, the target sample collection portion being configured to collect the target microparticles that have been sorted.

[11]

The microparticle sorting device according to any one of

[8] to [10], in which the microparticle sorting kit further includes a pre-sample accommodation portion configured to accommodate the sample liquid and located upstream of the sample liquid accommodation container, and has no filter portion between the pre-sample accommodation portion and the sample liquid accommodation container.

[12]

The microparticle sorting device according to any one of [8] to [11], in which the microparticle sorting kit has no filter portion between the sample liquid accommodation container and the microchip.

[13]

The microparticle sorting device according to any one of [8] to [12], further including:
a chip insertion unit into which the microchip is inserted;
a light irradiation unit configured to irradiate the microparticles flowing through the main channel with light;
a light detection unit configured to detect scattered light and/or fluorescence emitted from the microparticles; and
a control unit configured to control a traveling direction of the microparticles flowing through the main channel on the basis of data detected by the light detection unit.
   [14]

The microparticle sorting device according to any one of [1] to [13], in which the microparticles include bioparticles.

[15]

The microparticle sorting device according to [14], in which the bioparticles include cells.

[16]

A microparticle sorting kit including a microchip, the microchip including: a sample liquid inlet into which a sample liquid containing microparticles accommodated in a sample liquid accommodation container is introduced; a main channel through which the sample liquid that has been introduced flows; and a sorting channel where target microparticles are sorted from the sample liquid flowing through the main channel,
the microparticle sorting kit being configured to allow the sample liquid accommodation container to continuously connect to the microchip in a detachable manner.
   [17]

The microparticle sorting kit according to [16], configured to allow a target sample collection portion to continuously connect to the microchip in a detachable manner, the target sample collection portion being configured to collect the target microparticles that have been sorted.

[18]

The microparticle sorting kit according to [16] or [17], further including a pre-sample accommodation portion configured to accommodate the sample liquid and located upstream of the sample liquid accommodation container, and having no filter portion between the pre-sample accommodation portion and the sample liquid accommodation container.

[19]

The microparticle sorting kit according to any one of [16] to [18], having no filter portion between the sample liquid accommodation container and the microchip.

### REFERENCE SIGNS LIST

1 Sample liquid accommodation container
100 Microchip
200 Microparticle sorting kit
2011 Pre-sample accommodation portion
202, 202a, 202b Filter portion
203 Target sample collection portion
301 Chip insertion unit
302 Light irradiation unit
303 Light detection unit
304 Control unit
500 Sample liquid stirring device
510 First holding portion
520 Rotation portion
600 Microparticle sorting device
610 Kit attachment surface
620 Storage portion
621 Top plate portion
622 Top plate opening
623 Lid plate portion
623a First surface of lid plate portion
623b Second surface of lid plate portion
625 Space
630 Second holding portion

1. A microparticle sorting device comprising:
a kit attachment surface to which a microparticle sorting kit is detachably attached;
a storage portion including a top plate portion having a top plate opening and a lid plate portion having a first surface and capable of opening and closing the top plate opening, the storage portion having a space therein;
a first holding portion capable of holding a sample liquid accommodation container configured to accommodate a sample liquid containing microparticles; and
a second holding portion capable of holding a container, wherein
the first holding portion is provided in the space of the storage portion,
the second holding portion is provided on the first surface of the lid plate portion, and
the lid plate portion is capable of closing the top plate opening with the first surface and the second holding portion facing the space of the storage portion.

2. The microparticle sorting device according to claim 1, wherein the lid plate portion is separable from the top plate opening.

3. The microparticle sorting device according to claim 1, wherein the first holding portion is detachably provided in the space of the storage portion.

4. The microparticle sorting device according to claim 1, further comprising
a sample liquid stirring device located in the space of the storage portion, wherein
the first holding portion is provided in the sample liquid stirring device, and
the sample liquid stirring device includes a rotation portion configured to cause the first holding portion to perform a horizontal circular motion.

5. The microparticle sorting device according to claim 4, wherein the first holding portion is detachably provided in the sample liquid stirring device.

6. The microparticle sorting device according to claim 1, wherein the second holding portion is capable of holding a plurality of the containers.

7. The microparticle sorting device according to claim 1, wherein the second holding portion is capable of holding a plurality of the containers having different capacities.

8. The microparticle sorting device according to claim 1, wherein
the microparticle sorting kit includes a microchip, the microchip including: a sample liquid inlet into which the sample liquid containing the microparticles accommodated in the sample liquid accommodation container is introduced; a main channel through which the sample liquid that has been introduced flows; and a sorting channel where target microparticles are sorted from the sample liquid flowing through the main channel.

9. The microparticle sorting device according to claim 8, wherein the microparticle sorting kit is configured to allow the sample liquid accommodation container to continuously connect to the microchip in a detachable manner.

10. The microparticle sorting device according to claim 8, wherein the microparticle sorting kit is configured to allow a target sample collection portion to continuously connect to the microchip in a detachable manner, the target sample collection portion being configured to collect the target microparticles that have been sorted.

11. The microparticle sorting device according to claim 8, wherein the microparticle sorting kit further includes a pre-sample accommodation portion configured to accommodate the sample liquid and located upstream of the sample liquid accommodation container, and has no filter portion between the pre-sample accommodation portion and the sample liquid accommodation container.

12. The microparticle sorting device according to claim 8, wherein the microparticle sorting kit has no filter portion between the sample liquid accommodation container and the microchip.

13. The microparticle sorting device according to claim 8, further comprising:
a chip insertion unit into which the microchip is inserted;
a light irradiation unit configured to irradiate the microparticles flowing through the main channel with light;
a light detection unit configured to detect scattered light and/or fluorescence emitted from the microparticles; and
a control unit configured to control a traveling direction of the microparticles flowing through the main channel on a basis of data detected by the light detection unit.

14. The microparticle sorting device according to claim 1, wherein the microparticles include bioparticles.

15. The microparticle sorting device according to claim 14, wherein the bioparticles include cells.

16. A microparticle sorting kit comprising a microchip, the microchip including: a sample liquid inlet into which a sample liquid containing microparticles accommodated in a sample liquid accommodation container is introduced; a main channel through which the sample liquid that has been introduced flows; and a sorting channel where target microparticles are sorted from the sample liquid flowing through the main channel,
the microparticle sorting kit being configured to allow the sample liquid accommodation container to continuously connect to the microchip in a detachable manner.

17. The microparticle sorting kit according to claim 16, configured to allow a target sample collection portion to continuously connect to the microchip in a detachable manner, the target sample collection portion being configured to collect the target microparticles that have been sorted.

18. The microparticle sorting kit according to claim 16, further comprising a pre-sample accommodation portion configured to accommodate the sample liquid and located upstream of the sample liquid accommodation container, and having no filter portion between the pre-sample accommodation portion and the sample liquid accommodation container.

19. The microparticle sorting kit according to claim 16, having no filter portion between the sample liquid accommodation container and the microchip.
